(19) United States
(12) Patent Application Publication
(10) Pub. No.: US 2025/0072958 A1
(43) Pub. Date: Mar. 6, 2025

(54) POLYIMIDE LINED NITINOL CATHETER SHAFT

(71) Applicant: Medtronic Ireland Manufacturing Unlimited Company, Dublin (IE)

(72) Inventors: Jaclyn N. Kawwas, Philadelphia, PA (US); Jonathan Ashley Cope, Santa Rosa, CA (US); Alexandra C. Dotti, Rohnert Park, CA (US)

(21) Appl. No.: 18/722,358

(22) PCT Filed: Dec. 20, 2022
(86) PCT No.: PCT/EP2022/087064

§ 371 Date: Jun. 20, 2024

Related U.S. Application Data
(60) Provisional application No. 63/265,892, filed on Dec. 22, 2021.

(51) Int. Cl. A61B 18/14; A61B 18/00

(52) U.S. Cl. CPC A61B 18/1492; A61B 2018/00083; A61B 2018/00434

(57) ABSTRACT

In some examples, a catheter system includes an elongate shaft including a distal portion and a proximal portion and a neuromodulation element on the distal portion of the elongate shaft. The proximal portion of the shaft can include a proximal hypotube segment defining an inner lumen, and an electrically insulative tube located at least partially within the inner lumen of the proximal hypotube segment, the electrically insulative tube being attached to the proximal hypotube segment at at least one of a proximal portion or a distal portion of the proximal hypotube segment. The catheter system can also include one or more electrical leads extending distally within the inner lumen of the proximal hypotube segment and the electrically insulative tube to the neuromodulation element on the distal portion of the elongate shaft.

### TECHNICAL FIELD

The present technology is related to catheters, such as, for example, neuromodulation catheters including neuromodulation elements configured to deliver energy to nerves at or near a treatment location within a body lumen.

### BACKGROUND

The sympathetic nervous system (SNS) is a primarily involuntary bodily control system typically associated with stress responses. Fibers of the SNS extend through tissue in almost every organ system of the human body and can affect characteristics such as pupil diameter, gut motility, and urinary output. Such regulation can have adaptive utility in maintaining homeostasis or in preparing the body for rapid response to environmental factors. Chronic activation of the SNS, however, is a common maladaptive response that can drive the progression of many disease states. Excessive activation of the renal SNS, in particular, has been identified experimentally and in humans as a likely contributor to the complex pathophysiologies of hypertension, states of volume overload (e.g., heart failure), and progressive renal disease.

Sympathetic nerves of the kidneys terminate in the renal blood vessels, the juxtaglomerular apparatus, and the renal tubules, among other structures. Stimulation of the renal sympathetic nerves can cause, for example, increased renin release, increased sodium reabsorption, and reduced renal blood flow. These and other neural-regulated components of renal function are considerably stimulated in disease states characterized by heightened sympathetic tone. For example, reduced renal blood flow and glomerular filtration rate as a result of renal sympathetic efferent stimulation is likely a cornerstone of the loss of renal function in cardio-renal syndrome (i.e., renal dysfunction as a progressive complication of chronic heart failure). Pharmacologic strategies to thwart the consequences of renal sympathetic stimulation include centrally-acting sympatholytic drugs, beta blockers (e.g., to reduce renin release), angiotensin-converting enzyme inhibitors and receptor blockers (e.g., to block the action of angiotensin II and aldosterone activation consequent to renin release), and diuretics (e.g., to counter renal sympathetic mediated sodium and water retention). These pharmacologic strategies, however, have significant limitations including limited efficacy, compliance issues, side effects, and others.

### SUMMARY

The present disclosure is directed to catheters. Some examples are directed to neuromodulation catheters and techniques for assembling neuromodulation catheters. A catheter (e.g., an ablation catheter) may include a number of different elements that take time to assemble. In some examples, a catheter may include a neuromodulation element comprising one or more electrodes, where each of the one or more electrodes is coupled to an electrical lead (e.g., an electrically conductive wire). The catheter may further include an intermediate shaft, a proximal hypotube segment, and an electrically insulative tube located at least partially within an inner lumen of the proximal hypotube segment. To simplify assembly and/or reduce assembly time, the electrically insulative tube may be attached within the inner lumen of the proximal hypotube segment prior to the electrical lead(s) being inserted into the composite hypotube/insulative tube component. In this way, during an assembly process, the one or more electrical leads may be fed through multiple lumens simultaneously, which may simplify assembly and reduce assembly time of the catheter. For example, the one or more electrical leads may be fed through the electrically insulative tube and the proximal hypotube at the same time when the insulative tube is already located within the inner lumen of the proximal hypotube. This may simplify and/or reduce the number of steps during assembly, e.g., as compared to a process in which the one or more leads are first fed through an insulative tube and then feeding the combination of the one or more leads and the insulative tube through an inner lumen of a proximal hypotube.

In some examples a catheter system includes an elongate shaft including a distal portion and a proximal portion, and a neuromodulation element on the distal portion of the elongate shaft. The proximal portion of the elongate shaft can include: a proximal hypotube segment defining an inner lumen; and an electrically insulative tube located at least partially within the inner lumen of the proximal hypotube segment, the electrically insulative tube being attached to the proximal hypotube segment at at least one of a proximal portion or a distal portion of the proximal hypotube segment. The catheter system can also include one or more electrical leads extending distally within the inner lumen of the proximal hypotube segment and the electrically insulative tube to the neuromodulation element on the distal portion of the elongate shaft.

In some examples, a method of assembling a catheter includes: attaching an electrically insulative tube at at least one of a proximal portion or a distal portion of a proximal hypotube segment of an elongate shaft of the catheter, wherein the electrically insulative tube is located at least partially within an inner lumen defined by the proximal hypotube segment; and feeding one or more electrical leads from a distal portion of the elongate shaft proximally through the elongate shaft, including through an inner lumen defined by the electrically insulative tube and the inner lumen defined by the proximal hypotube segment.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

Reference is made to the attached drawings, wherein elements having the same or similar reference numeral designations represent similar elements throughout.

FIG. 1 is a partially schematic perspective view illustrating a therapeutic system configured in accordance with an example of the present technology. The system is shown in FIG. 1 includes a neuromodulation catheter having a shaft.

FIG. 2 is an exploded profile view of the catheter shown in FIG. 1.

FIG. 3 is an enlarged exploded profile view of portions of the catheter shown in FIG. 1 taken at respective locations designated in FIG. 2.

FIG. 4 is a perspective view of a distal skived segment of a proximal hypotube segment of a neuromodulation catheter configured in accordance with an example of the present technology.

FIG. 5 is a perspective view of a proximal assembly of a neuromodulation catheter, as well as a proximal portion of a proximal hypotube segment and electrically insulative tube in accordance with an example of the present technology.

FIG. 6 is a profile view of a proximal hypotube segment and a proximal jacket of the shaft shown in FIG. 1.

FIG. 7 is a cross-sectional profile view of the proximal hypotube segment and the proximal jacket shown in FIG. 6 taken along a line 8-8 designated in FIG. 6.

FIG. 8 is an enlarged profile view of a skived segment of the proximal hypotube segment and the proximal jacket shown in FIG. 6 taken at a location designated in FIG. 6.

FIG. 9A is a cross sectional view of an example intermediate segment distal to a rapid exchange joint, including electrical leads and a guidewire lumen, in accordance with one or more examples of the present disclosure.

FIG. 9B is a cross sectional view of an example proximal hypotube segment proximal to a rapid exchange joint, including electrical leads extending through an electrically insulative tube, in accordance with one or more examples of the present disclosure.

FIG. 10 is a side view of example components of a catheter in an unassembled state, including a proximal hypotube segment 628, and an intermediate segment 640 in accordance with one or more examples of the present disclosure.

FIG. 11 is a side view of example partially assembled catheter components, including a distal assembly and electrical leads, in accordance with one or more examples of the present disclosure.

FIG. 12 is a side view of an example assembled catheter, in accordance with one or more examples of the present disclosure.

FIG. 13 is a flow chart illustrating an example method for assembling components of a neuromodulation catheter, in accordance with one or more examples of the present disclosure.

FIG. 14 is a flow chart illustrating another example method for assembling components of a neuromodulation catheter, in accordance with one or more examples of the present disclosure.

FIGS. 15A and 15B are example side views of a proximal hypotube segment and proximal outer jacket, in accordance with one or more examples of the present disclosure.

FIG. 16 is an example side view of a neuromodulation catheter, including an elongate shaft and proximal assembly, in accordance with one or more examples of the present disclosure.

FIG. 17 is a side view of the example proximal assembly of FIG. 16, in accordance with one or more examples of the present disclosure.

### DETAILED DESCRIPTION

The present technology is directed to neuromodulation catheters and techniques for assembling a neuromodulation catheter. Although examples are described primarily with respect to renal neuromodulation, a person having ordinary skill in the art reading this description will understand that the devices, systems, and methods described herein may be used for neuromodulation at any suitable location within a body of a patient, including intravascular locations. Exemplifying vascular locations include, for example, a renal artery, external iliac artery, internal iliac artery, internal pudendal artery, celiac artery, mesenteric artery, superior mesenteric artery, inferior mesenteric artery, hepatic artery, splenic artery, gastric artery, left gastric artery, pancreatic artery, uterine artery, ovarian artery, testicular artery, and/or their associated arterial branches, accessories, veins, etc.

As used herein, the terms “distal” and “proximal” define a position or direction with respect to the treating clinician or clinician's control device (e.g., a handle assembly). “Distal” or “distally” can refer to a position distant from or in a direction away from the clinician or clinician's control device. “Proximal” and “proximally” can refer to a position near or in a direction toward the clinician or clinician's control device.

Neuromodulation, such as denervation, may be used to modulate activity of one or more nerves and may be used to affect activity of the sympathetic nervous system (SNS). Renal neuromodulation, for example, may be used to modulate activity of one or more renal nerves and may be used to affect activity of the SNS. In renal neuromodulation, one or more therapeutic elements may be introduced near renal nerves located between an aorta and a kidney of a patient. In some examples, the one or more therapeutic elements may be carried by or attached to a catheter, and the catheter may be introduced intravascularly, e.g., into a renal artery via a brachial artery, femoral artery, or radial artery approach. In other examples, the one or more therapeutic elements may be introduced extravascularly, e.g., using a laparoscopic technique.

Neuromodulation may be accomplished using one or more of a variety of treatment modalities, including electrical stimulation, radio frequency (RF) energy, microwave energy, ultrasound energy, a chemical agent, thermal energy (e.g., cryoablation or direct heating) or the like. Although examples are described primarily with respect to the RF modality, a person having ordinary skill in the art reading this description will understand that the devices, systems, and methods described herein also may be used for neuromodulation employing other modalities, including those referenced herein. In some examples, an RF ablation system includes an RF generator configured to generate RF energy and deliver RF energy to tissue via one or more electrodes carried by a catheter and positioned within a lumen of a body of a patient. For example, the lumen may be a vessel, such as a vein or artery. In some examples, the lumen may be a renal artery, such as a main renal artery, an accessory renal artery, a branch vessel, or the like. The RF energy may heat tissue to which the RF energy is directed (which tissue includes one or more renal nerves) and modulate the activity of the one or more renal nerves.

A neuromodulation catheter (e.g., for performing renal denervation) may include a number of steps for assembly. The higher the number of steps, generally the longer it may take to assemble the neuromodulation catheter. Accordingly, it may be desirable to reduce the number of steps for assembly of the neuromodulation catheter. Example methods for assembling neuromodulation catheters may include: feeding electrical leads of a distal neuromodulation assembly through an electrically insulative tube, feeding the electrical leads and electrically insulative tube through an intermediate segment of an elongate shaft of the catheter, feeding the electrical leads and electrically insulative tube through a proximal hypotube segment of the elongate shaft of the catheter, and attaching the electrically insulative tube to the proximal hypotube segment.

In examples of the current disclosure, a neuromodulation catheter may include fewer steps for assembly, and thus take less time to assemble. For example, a method for assembling a neuromodulation catheter (e.g., an RF ablation catheter) may include attaching an electrically insulative tube to a proximal hypotube segment of an elongate shaft of the catheter, aligning an intermediate segment of the elongate shaft with the proximal hypotube and electrically insulative tube combination, and feeding electrical leads of a distal neuromodulation assembly through inner lumens of the intermediate segment and the proximal hypotube/electrically insulative tube combination. In examples of the current disclosure, instead of feeding the electrical leads through multiple inner lumens individually, attaching the electrically insulative tube to the proximal hypotube segment first permits the electrical leads to be fed through the intermediate segment, proximal hypotube segment, and electrically insulative tube at once. In this way, the proximal hypotube segment may hold the electrically insulative tube rigidly in place during assembly, and the assembly of neuromodulation catheters may be simpler and less time intensive. Electrically insulative tube may be made of a polymer that is not rigid enough to easily handle by itself. Attaching the electrically insulative tube to the proximal hypotube segment before feeding electrical leads through the electrically insulative tube or the proximal hypotube prevents kinking, ripping, rolling, and/or crushing of the electrically insulative tube during assembly. Attaching the electrically insulative tube to the proximal hypotube segment before feeding electrical leads through the electrically insulative tube or the proximal hypotube also reduces the time of forming an exchange joint between the proximal hypotube and the intermediate segment, as the electrically insulative tube is bonded in place and does not shift during exchange joint formation. The examples of the current disclosure may result in time reductions of, e.g., up to seventy percent or greater for assembly of the neuromodulation catheter.

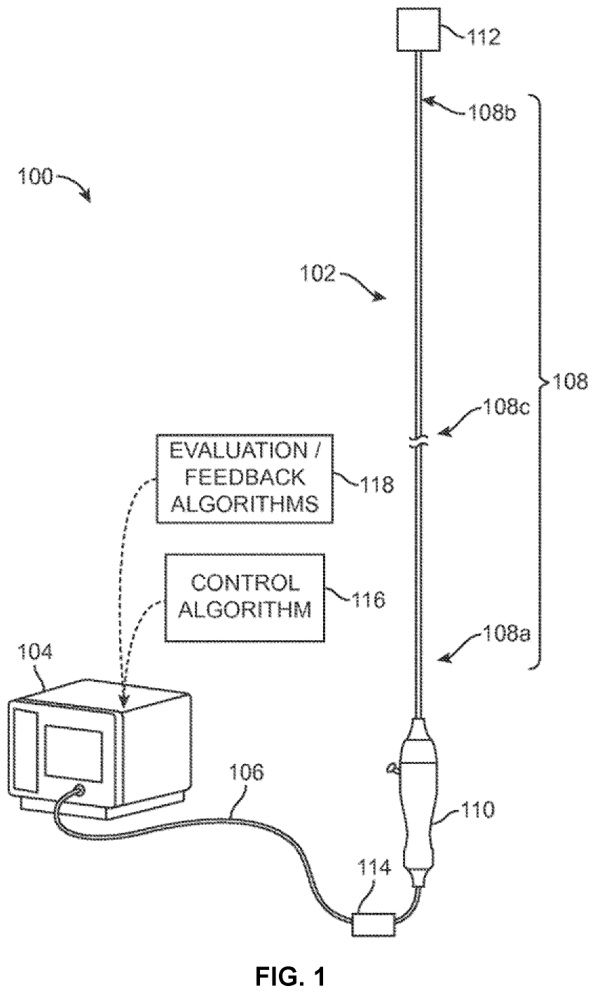

FIG. 1 is a partially schematic perspective view illustrating a therapeutic system 100 including a neuromodulation catheter 102 configured in accordance with some examples of the present disclosure. Therapeutic system 100 includes a neuromodulation catheter 102, an RF generator 104, and a cable 106 extending between catheter 102 and RF generator 104. Neuromodulation catheter 102 includes an elongate shaft 108 (also referred to as an elongate body 108) having a proximal portion 108a, a distal portion 108b, and an intermediate portion 108c between proximal portion 108a and distal portion 108b. Neuromodulation catheter 102 may further include a handle 110 (e.g., a proximal assembly) operably connected to elongate shaft 108 via proximal portion 108a and a neuromodulation element 112 (shown schematically in FIG. 1) that is part of or attached to distal portion 108b. Elongate shaft 108 is configured to locate the neuromodulation element 112 (e.g., a distal assembly) at a treatment location within or otherwise proximate to a body lumen (e.g., a blood vessel, a duct, an airway, or another naturally occurring lumen within the human body). In some examples, elongate shaft 108 is configured to locate neuromodulation element 112 at an intraluminal (e.g., intravascular) location. Neuromodulation element 112 may be configured to provide or support a neuromodulation treatment at the treatment location. Elongate shaft 108 and neuromodulation element 112 may measure 2, 3, 4, 5, 6, or 7 French or other suitable sizes.

Intraluminal delivery of neuromodulation catheter 102 may include percutaneously inserting a guidewire (not shown) into a body lumen of a patient and moving elongate shaft 108 and neuromodulation element 112 (e.g., a distal assembly) along the guidewire until neuromodulation element 112 reaches a suitable treatment location. Alternatively, neuromodulation catheter 102 may be a steerable or non-steerable device configured for use without a guidewire. Additionally, or alternatively, neuromodulation catheter 102 may be configured for use with another type of guide member, such as a guide catheter or a sheath (not shown), alone, or in addition to a guidewire.

RF generator 104 is configured to control, monitor, supply, and/or otherwise support operation of neuromodulation catheter 102. In other examples, neuromodulation catheter 102 may be self-contained or otherwise configured for operation independent of RF generator 104. When present, RF generator 104 is configured to generate a selected form and/or magnitude of RF energy for delivery to tissue at a treatment location via neuromodulation element 112. For example, RF generator 104 may be configured to generate RF energy (e.g., monopolar and/or bipolar RF energy). In other examples, RF generator 104 may be another type of device configured to generate and deliver another suitable type of energy to neuromodulation element 112 for delivery to tissue at a treatment location via neuromodulation element 112.

Along cable 106 or at another suitable location within therapeutic system 100, therapeutic system 100 may include a control device 114 configured to initiate, terminate, and/or adjust operation of one or more components of neuromodulation catheter 102 directly and/or via RF generator 104. RF generator 104 may be configured to execute an automated control algorithm 116 and/or to receive control instructions from an operator. Similarly, in some implementations, RF generator 104 is configured to provide feedback to an operator before, during, and/or after a treatment procedure via an evaluation/feedback algorithm 118.

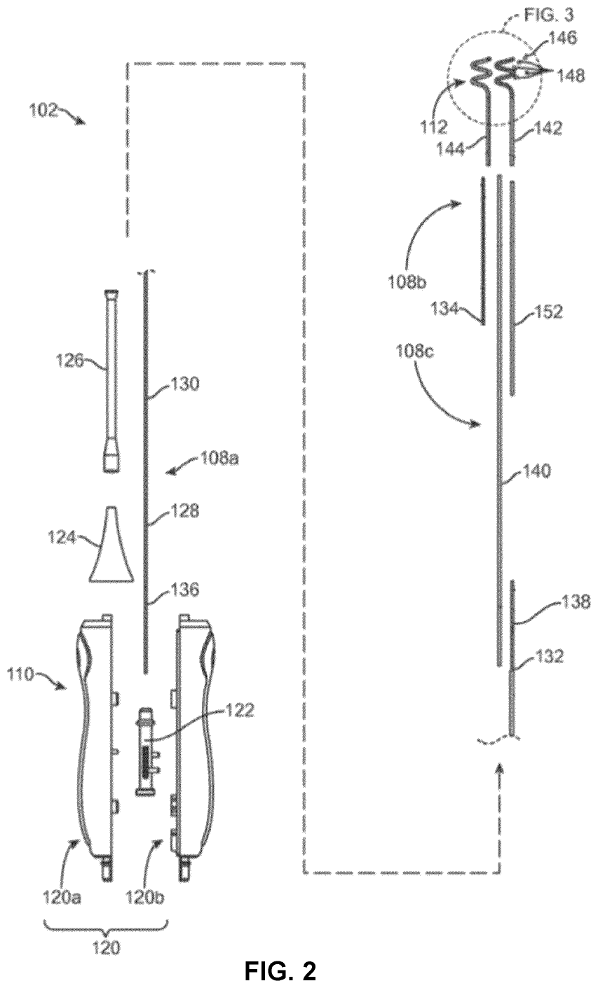

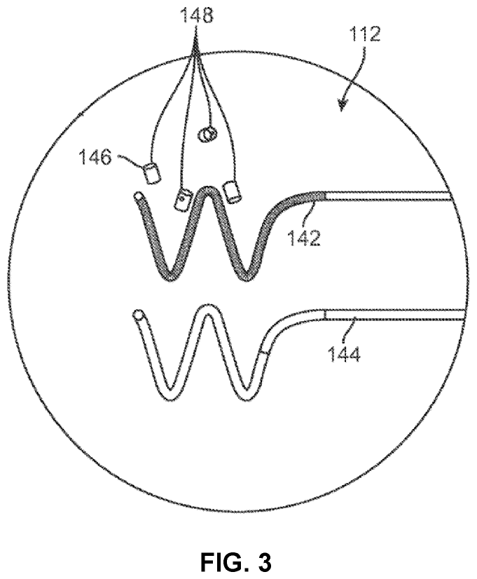

FIG. 2 is an exploded profile view of neuromodulation catheter 102. FIG. 3 is an enlarged exploded profile view of portions of neuromodulation catheter 102 taken at the location designated in FIG. 2. With reference to FIG. 2, handle 110 may include mating shell segments 120 (individually identified as shell segments 120a, 120b) and a connector 122 (e.g., a luer connector) operably positioned between the mating shell segments 120. Handle 110 may further include a distally tapered strain-relief element 124 operably connected to distal ends of shell segments 120. Slidably positioned over elongate shaft 108, neuromodulation catheter 102 may include a loading tool 126 configured to facilitate loading catheter 102 onto a guidewire (not shown).

Elongate shaft 108 may include an assembly of parallel tubular segments. At proximal portion 108a, elongate shaft 108 may include a proximal hypotube segment 128, a proximal outer jacket 130, and a first electrically insulative tube 132. Proximal outer jacket 130 may be a polymer outer jacket disposed around at least a portion of an outer surface of proximal hypotube segment 128. For example, proximal outer jacket may include a polyether block amide (e.g., PEBAX® 7233, PEBAX® 7233 with twenty percent weight siloxane, or the like). First electrically insulative tube 132 may be an electrically insulative polymer tube or coating located at least partially within an inner lumen of proximal hypotube segment 128. First electrically insulative tube 132 may be attached to proximal hypotube segment 128 at at least one of a proximal portion or a distal portion of proximal hypotube segment 128. First electrically insulative tube 132 may define an inner lumen, and one or more electrical leads (e.g., electrically conductive wires 148) may be positioned within the inner lumen of first electrically insulative tube 132. First electrically insulative tube 132 may electrically insulate the electrical leads from proximal hypotube segment 128. The one or more electrical leads may extend distally within the inner lumen of proximal hypotube segment 128 and electrically insulative tube 132 to neuromodulation element 112 on the distal portion of the elongate shaft (e.g., to one or more electrodes 146 of neuromodulation element 112).

Proximal hypotube segment 128 may include a proximal portion, a proximal end, and a first inner lumen. When assembled, handle 110 (e.g., proximal assembly) may define a second inner lumen, wherein the proximal portion of proximal hypotube segment 128 is received within the second inner lumen of the handle 110, and the proximal end of proximal hypotube segment 128 terminates within the second inner lumen. For example, when assembled, elongate shaft 108 may extend through coaxial lumens (also not shown) of strain-relief element 124 and loading tool 126, respectively, and between shell segments 120 to connector 122. Proximal hypotube segment 128 may define a length. In some examples, the length may be longer or shorter to allow for different types of access to a patient's body. In some examples, proximal hypotube segment 128 and/or elongate shaft 108 may be longer for radial access to a patient's body than proximal hypotube segment 128 and/or elongate shaft 108 for femoral access to a patient's body. In some examples of an elongate shaft 108 for femoral access to a patient's body, a combined length of proximal hypotube segment 128 and an intermediate segment of elongate shaft 108 may be around 31.86 inches long. In some examples of an elongate shaft 108 for radial access to a patient's body, a combined length of proximal hypotube segment 128 and an intermediate segment of elongate shaft 108 may be around 55.62 inches long. Proximal hypotube segment 128 and the intermediate segment may be made predominantly from nitinol, stainless steel, or other suitable materials.

With reference again to FIGS. 2 and 3, first electrically insulative tube 132 may extend distally along a longitudinal axis of proximal hypotube segment 128 into skived segment 138 of proximal hypotube 128. In some examples, a distal end of first electrically insulative tube 132 may extend distally beyond distal skived segment 138 of proximal hypotube segment 128. In some examples, a distal end of first electrically insulative tube 132 may terminate within a portion of skived segment 138. In some examples, a distal end of first electrically insulative tube 132 may terminate within a portion of proximal hypotube segment 128 proximal to skived segment 138. First electrically insulative tube 132 may extend proximally along the longitudinal axis beyond a proximal end of proximal hypotube segment 128. For example, a proximal end of first electrically insulative tube 132 may terminate within the second inner lumen defined by handle 110, proximal to the proximal end of proximal hypotube segment 128. In some examples, the proximal end of first electrically insulative tube 132 may terminate proximal to a proximal end of the second inner lumen defined by handle 110.

First electrically insulative tube 132 may be attached to proximal hypotube segment 128 at at least one of a proximal portion or a distal portion of proximal hypotube segment 128. In some examples, first electrically insulative tube 132 may be attached to distal skived segment 138 with a reflowed polymer, adhesive, or the like. In some examples, first electrically insulative tube 132 may be attached to the proximal portion of proximal hypotube segment 128 with a reflowed polymer, adhesive, or the like. In some examples, first electrically insulative tube 132 may be attached to a proximal portion of handle 110 with a reflowed polymer, adhesive, or the like. In some examples, first electrically insulative tube 132 may be friction fit onto an inner diameter of the inner lumen of proximal hypotube segment 128. For example, first electrically insulative tube 132 may be cooled or otherwise shrunk before being positioned within the inner lumen of proximal hypotube segment 128. After returning to normal temperature, first electrically insulative tube 132 may expand to press against inner walls of the inner lumen of proximal hypotube segment 128. In some examples, first electrically insulative tube 132 is melted onto an inner diameter of the proximal hypotube segment to attach the electrically insulative tube to the proximal hypotube segment. First electrically insulative tube 132 may be melted onto a proximal portion, distal portion, and/or at one or more of a plurality of locations of the inner walls of the inner lumen of proximal hypotube segment 128. In some examples, an adhesive may be applied between an outer diameter of first electrically insulative tube 132 and the inner diameter of proximal hypotube segment 128 to attach first electrically insulative tube 132 to proximal hypotube segment 128. An adhesive may be applied between an outer diameter of first electrically insulative tube 132 and the inner diameter of proximal hypotube segment 128 at a proximal portion, distal portion, and/or at one or more of a plurality of locations of the inner walls of the inner lumen of proximal hypotube segment 128.

Elongate shaft 108 may include an intermediate segment 140 beginning proximally at a region of elongate shaft 108 at which first electrically insulative tube 132 distally emerges from proximal hypotube segment 128. Intermediate segment 140 may be more flexible than proximal hypotube segment 128. Intermediate segment 140 may be attached at one end to the distal assembly (e.g., neuromodulation element 112) and attached at the other end to proximal hypotube segment 128 via a rapid exchange joint. At the region of elongate shaft 108 at skived segment 138, intermediate segment 140 may be coaxially aligned with proximal hypotube segment 128 so that a rapid exchange joint may be formed. From this region, intermediate segment 140 may extend distally to distal end portion 108b of shaft 108. At a distal end of intermediate segment 140, elongate shaft 108 may be operably connected to neuromodulation element 112 (e.g., a distal assembly). Intermediate segment 140 may be attached to the distal assembly or proximal hypotube segment 128 using reflowed polymer, an adhesive, or the like. For example, intermediate segment 140 may be attached to the distal assembly or proximal hypotube segment 128 using a polyether block amide (e.g., PEBAX®, commercially available from Arkema Group of Colombes, France). In addition to, or instead of, PEBAX®, intermediate segment may be attached to the distal assembly or proximal hypotube segment 128 using another reflowable polymer. The one or more electrical leads may extend distally within an inner lumen of intermediate segment 140 to neuromodulation element 112 on distal portion 108b of elongate shaft 108 (e.g., to electrodes 146 of neuromodulation element 112).

A proximal end of guidewire tube 134 may be positioned in the rapid exchange joint to allow a guidewire to pass from the inside of elongate shaft 108 distal to the rapid exchange joint to the outside of elongate shaft 108 proximal of the rapid exchange joint. A proximal portion of guidewire tube 134, including the proximal end of guidewire tube 134 may be attached to elongate shaft 108 using reflowed polymer, an adhesive, or the like. The proximal portion of guidewire tube 134 may include a skived section configured to define a smooth diameter transition between proximal outer jacket 130 (e.g., a polymer outer jacket) and a location where a portion of guidewire tube 134 extends into the rapid exchange joint. The guidewire may be positioned inside an interior lumen of guidewire tube 134 in a distal portion 108b of elongate shaft 108, exit elongate shaft 108 at the rapid exchange joint, and be positioned outside elongate shaft 108 from the rapid exchange joint proximally of the rapid exchange joint (e.g., to at least handle 110). Guidewire tube 134 may extend distally from the rapid exchange joint to neuromodulation element 112.

In FIGS. 2 and 3, neuromodulation element 112 is shown in a radially expanded deployed state. Neuromodulation element 112 may be movable from a low-profile delivery state to the radially expanded deployed state. When neuromodulation element 112 is in the radially expanded deployed state, a distal shape-memory structure 142, such as a Nitinol-based structure (such as a helical hollow strand (HHS®) structure, available from Fort Wayne Metals, Fort Wayne, Indiana), or another shape-memory material, may have a shape that is more helical (spiral) than its shape when neuromodulation element 112 is in the low-profile delivery state. In at least some cases, distal shape-memory structure 142 has the more helical shape when at rest and is configured to be urged into the less helical shape by an external sheath (not shown), an internal guidewire, an internal mandrel, or the like. In at least some cases, the material of distal shape-memory structure 142 is electrically conductive. Accordingly, neuromodulation element 112 may include a second electrically insulative tube 152 disposed around an outer surface of distal shape-memory structure 142 so as to electrically separate electrodes 146 from distal shape-memory structure 142. In some examples, first and second electrically insulative tubes 132, 152 are made at least partially (e.g., predominantly or entirely) of polyimide and polyether block amide, respectively. In other examples, first and second electrically insulative tubes 132, 152 may be made of other suitable materials, e.g., polyurethane, polyester, other suitable polymers or predominantly polymer materials. Distal jacket 144 may be tubular and configured to be disposed around at least a portion of an outer surface of distal shape-memory structure 142.

Neuromodulation element 112 may include one or more electrodes 146 (e.g., one electrode, two electrodes, three electrodes, four electrodes, or the like) and may be configured to transform between a substantially straight delivery configuration and a deployed configuration (e.g., a spiral or helical configuration). Neuromodulation catheter 102 may further include one or more wires 148 or wire pairs extending from a proximal end (or from near the proximal end) of neuromodulation catheter 102 to the one or more electrode(s) 146 at distal portion 108b of neuromodulation catheter 102, each wire (or wire pair) of one or more wires 148 being electrically coupled (e.g., welded or otherwise affixed) to a corresponding electrode of one or more electrodes 146 to deliver energy, and in some examples, to form a thermocouple for conducting temperature measurements. Distal portion 108b of neuromodulation catheter 102 may also define one or more openings or slots through which the one or more wire(s) 148 extend in order to contact and electrically couple to the one or more electrode(s) 146.

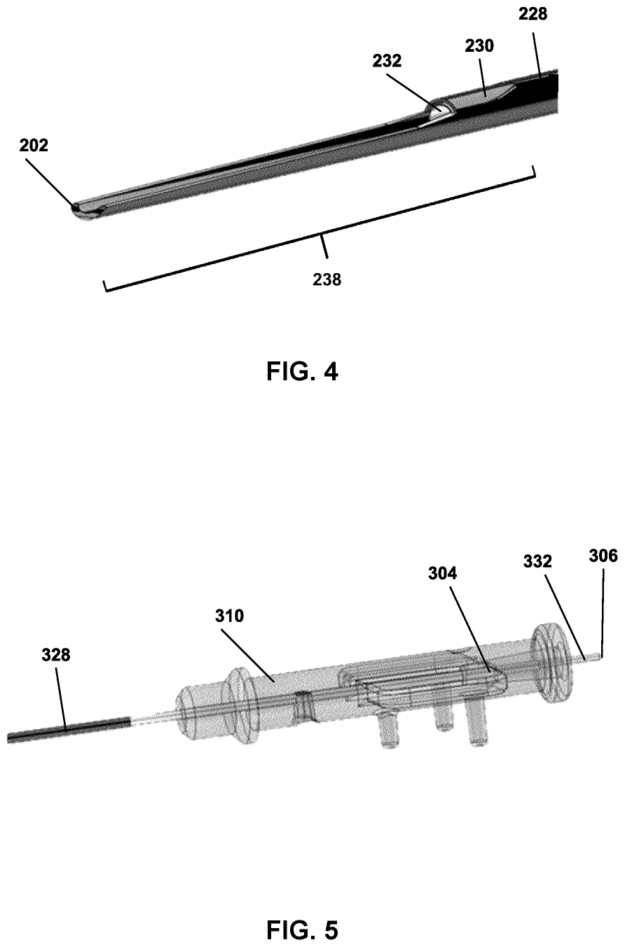

FIG. 4 is a perspective view of a distal skived segment 238 of a proximal hypotube segment 228 of a neuromodulation catheter configured in accordance with an example of the present technology. Skived segment 238 may facilitate the formation of a rapid exchange joint between a distal portion of proximal hypotube segment 228 and an intermediate segment of the elongate shaft. Proximal hypotube segment 228 may be an example of proximal hypotube segment 128 of FIGS. 1-2. Skived segment 238 may be an example of skived segment 138 of FIGS. 1-2. Proximal outer jacket 230 may be an example of proximal outer jacket 130 of FIGS. 1-2. Electrically insulative tube 232 may be an example of first electrically insulative tube 132 of FIGS. 1-2. Proximal outer jacket 230 may be a polymer outer jacket disposed around at least a portion of an outer surface of proximal hypotube segment 228. Electrically insulative tube 232 may be an electrically insulative polymer tube or coating located at least partially within an inner lumen of proximal hypotube segment 228. Skived segment 238 may be configured to define a smooth diameter transition between proximal outer jacket 230 (e.g., a polymer outer jacket) and a location where a portion of a guidewire tube extends into the rapid exchange joint. For example, skived segment 238 may be deburred, tapered, and sand blasted to ensure the smooth transition. A distal portion of electrically insulative tube 232 and a distal portion of proximal outer jacket 230 may be skived to define the smooth diameter transition as well. Electrically insulative tube 232 may be attached to skived segment 238 and proximal outer jacket 230 with a reflowed polymer, adhesive, or the like.

Proximal outer jacket 230 and electrically insulative tube 232 may extend distally into skived segment 238 (e.g., with the distal end of insulative tube 232 terminating at a point within skived segment 238 or extending beyond the distal end of skived segment 238). For example, electrically insulative tube 232 and/or proximal outer jacket 230 may extend 2.5 millimeters distally into skived segment 238, where skived segment 238 has a length longer than 2.5 millimeters.

In some examples, when in a fully assembled configuration, distal end 202 of proximal hypotube segment 228 may be positioned within an intermediate segment of the elongate shaft of the catheter to form the rapid exchange joint. In some examples, distal end 202 may be coaxially aligned with the intermediate segment to form the rapid exchange joint.

FIG. 5 is a perspective view of a proximal assembly 310 of a neuromodulation catheter, as well as a proximal portion of a proximal hypotube segment 328 and electrically insulative tube 332 in accordance with an example of the present technology. Proximal assembly 310 may be an example of handle 110 of FIGS. 1-2. Proximal hypotube segment 328 may be an example of proximal hypotube segment 128 of FIGS. 1-2. Electrically insulative tube 332 may be an example of first electrically insulative tube 132 of FIGS. 1-2. Proximal assembly 310 may define an inner lumen, wherein a proximal portion of proximal hypotube segment 328 may be received within the inner lumen of proximal assembly 310, and a first proximal end 304 of proximal hypotube segment 328 terminates within the inner lumen. A second proximal end 306 of electrically insulative tube 332 may extend proximally of proximal assembly 310 beyond a third proximal end of the inner lumen defined by proximal assembly 310. The portion of electrically insulative tube 332 proximal to proximal assembly 310 may act to protect electrical leads and/or thermocouple wires located within proximal assembly 310 and/or extending out of the proximal end of proximal assembly 310. For example, second proximal end 306 of electrically insulative tube 332 may extend four millimeters proximally of proximal assembly 310 beyond a third proximal end of the inner lumen defined by proximal assembly 310.

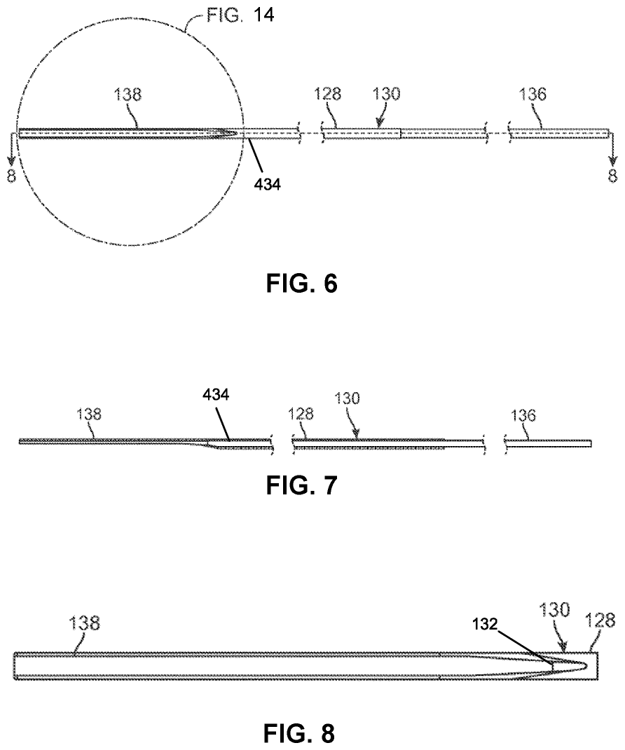

FIG. 6 is a profile view of proximal hypotube segment 128 and proximal outer jacket 130. FIG. 7 is a cross-sectional profile view of proximal hypotube segment 128 and proximal outer jacket 130 taken along a line 8-8 designated in FIG. 6. FIG. 8 is an enlarged profile view of a skived segment 138 of proximal hypotube segment 128 and proximal jacket 130 taken at a location designated in FIG. 6. As shown in FIGS. 6 and 7, proximal jacket 130 may be absent from the outer surface of the proximal hypotube segment 128 at a proximal portion 136 of proximal hypotube 128. This may be useful, for example, to facilitate connecting the proximal hypotube segment 128 to the connector 122 (shown in FIG. 2). In contrast, proximal outer jacket 130 may be disposed on at least a portion of the outer surface of proximal hypotube segment 128 and a portion of electrically insulative tube 132 at distal skived segment 138 in a distal portion 434 of proximal hypotube segment 128.

In some examples, proximal hypotube segment 128 is made at least partially (e.g., predominantly or entirely) of nitinol. In some examples, proximal hypotube segment 128 may be made at least partially of stainless steel or other suitable materials. In these and other examples, proximal outer jacket 130 may be made at least partially (e.g., predominantly or entirely) of a polymer blend including polyether block amide and polysiloxane. For example, the polymer blend may include greater than 15% by weight polysiloxane. In a particular example, the polymer blend includes about 20% by weight polyether block amide and about 80% by weight polyether block amide. This material may allow proximal outer jacket 130 to have sufficient lubricity for use without an outer coating, among other potential advantages. In still other examples, proximal hypotube segment 128 and proximal outer jacket 130 may be made of other suitable materials.

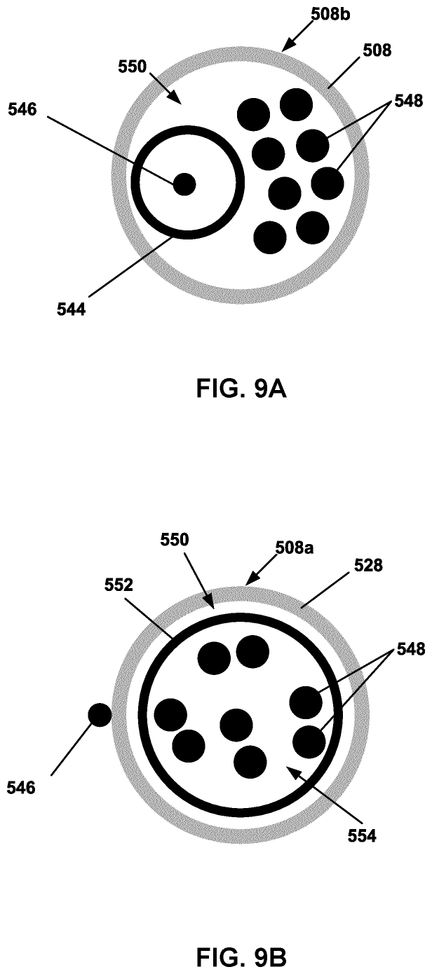

FIG. 9A is a cross sectional view of a distal portion 508b of elongate shaft 508 and other components of a catheter, where distal portion 508b is distal to a rapid exchange joint between an intermediate segment of elongate shaft 508 and a proximal hypotube segment of elongate shaft 508. Elongate shaft 508 may be an example of elongate shaft 108 of FIGS. 1-2. Guidewire tube 544 may be an example of guidewire tube 134 of FIGS. 1-2. Wires 548 may be an example of wires 148 of FIGS. 2-3. In distal portion 508b of elongate shaft 508, a guidewire tube 544 tube may be positioned within a lumen 550 of elongate shaft 508. A guidewire 546 may be positioned inside a guidewire lumen of guidewire tube 544. Furthermore, in distal portion 508b of elongate shaft 508, wires 548 may be positioned within lumen 550 of elongate shaft 508 alongside (e.g., substantially parallel to and outside of) guidewire tube 544.

The catheter may include one or more wires 548 (e.g., electrical leads) or wire pairs extending from a proximal end (or from near the proximal end) of the catheter to the electrode(s) at the distal portion of the catheter. One or more wires 548 may include one or more wires for each electrode at the distal portion of the catheter. For example, one or more wires 548 may include a first wire and a second wire for each electrode, such that the catheter includes twice the number of wires as electrodes. As another example, one or more wires 548 may include a single wire for each electrode, such that the catheter includes the same number of wires as electrodes. In implementations in which one or more wires 548 may include a first wire and a second wire for each electrode, each wire or wire pair may be electrically coupled to a corresponding electrode to deliver energy and act as a thermocouple for sensing temperature at the electrode. In some examples, wires 548 may be positioned inside a second electrically insulative tube as described with reference to FIGS. 2 and 3 in at least a portion of distal portion 508b. In some examples, wires 548 may include a core made of electrically conductive material (e.g., copper), and an electrically insulative layer surrounding the core to electrically isolate the conductive material of each wire from the other wires 548.

FIG. 9B is a cross sectional view of a proximal portion 508a of elongate shaft 508, where proximal portion 508a is proximal to a rapid exchange joint between an intermediate segment of elongate shaft 508 and a proximal hypotube segment of elongate shaft 508. Proximal hypotube segment 528 may be an example of proximal hypotube segment 128 of FIGS. 1-2. Electrically insulative tube 552 may be an example of first electrically insulative tube 132 of FIGS. 1-2. In proximal portion 508a of elongate shaft 508, the one or more wires 548 (e.g., electrical leads) may be positioned inside a first electrically insulative tube 552. First electrically insulative tube 552 may be an electrically insulative polymer (or other electrically insulative material) disposed within lumen 550 of proximal hypotube segment 528. Electrically insulative tube 552 may have an inner wall defining an inner diameter and an outer wall defining an outer diameter. For example, electrically insulative tube 552 may have an inner diameter of around 0.022 inches and an outer diameter of around 0.024 inches. The inner wall and outer wall of electrically insulative tube 552 may define a thickness of electrically insulative tube 552. In some examples, the thickness of electrically insulative tube 552 may range from 0.00025 inches to 0.004 inches. In some examples, a thickness of electrically insulative tube 552 before being attached to proximal hypotube segment 528 may be different than a thickness of electrically insulative tube 552 after being attached to proximal hypotube segment 528. Although FIG. 9B depicts a space between an inner wall of proximal hypotube segment 528 and an outer wall of first electrically insulative tube 552, in some examples, rather than being a separate tube, first electrically insulative tube 552 may instead be a coating or layer on an inner surface of proximal hypotube segment 528. First electrically insulative tube 552 may define a lumen 554, wherein one or more electrical leads (e.g., wires 548) are positioned within lumen 554 of first electrically insulative tube 552. First electrically insulative tube 552 may electrically insulate one or more wires 548 from the elongate shaft 508. In the example of FIG. 9B, guidewire 546 may be positioned on the outside of elongate shaft 508 proximal to the rapid exchange joint, as guidewire 546 may be extended through an opening at the rapid exchange joint.

First electrically insulative tube 552 may be attached to proximal hypotube segment 528 at at least one of a proximal portion or a distal portion of proximal hypotube segment 528. In some examples, first electrically insulative tube 552 may be attached to a distal skived segment of proximal hypotube segment 528 with a reflowed polymer, adhesive, or the like. In some examples, first electrically insulative tube 552 may be attached to a proximal portion of proximal hypotube segment 528 with a reflowed polymer, adhesive, or the like. In some examples, first electrically insulative tube 552 may be friction fit onto an inner diameter of the inner lumen of proximal hypotube segment 528. For example, first electrically insulative tube 552 may be cooled or otherwise shrunk before being positioned within the inner lumen of proximal hypotube segment 528. After returning to normal temperature, first electrically insulative tube 552 may expand to press against inner walls of the inner lumen of proximal hypotube segment 528. In some examples, first electrically insulative tube 552 may be melted onto an inner diameter of proximal hypotube segment 528. First electrically insulative tube 552 may be melted onto a proximal portion, distal portion, and/or at one or more of a plurality of locations of the inner walls of the inner lumen of proximal hypotube segment 528. In some examples, an adhesive may be applied between an outer diameter of first electrically insulative tube 552 and the inner diameter of proximal hypotube segment 528. An adhesive may be applied between an outer diameter of first electrically insulative tube 552 and the inner diameter of proximal hypotube segment 528 at a proximal portion, distal portion, and/or at one or more of a plurality of locations of the inner walls of the inner lumen of proximal hypotube segment 528.

The sizes of wires 548 and guidewire 546 in FIGS. 9A and 9B are depicted for ease of visibility only, and should not be limited to their size with respect to the portions 508a and 508b of elongate shaft 508 depicted in FIGS. 9A and 9B.

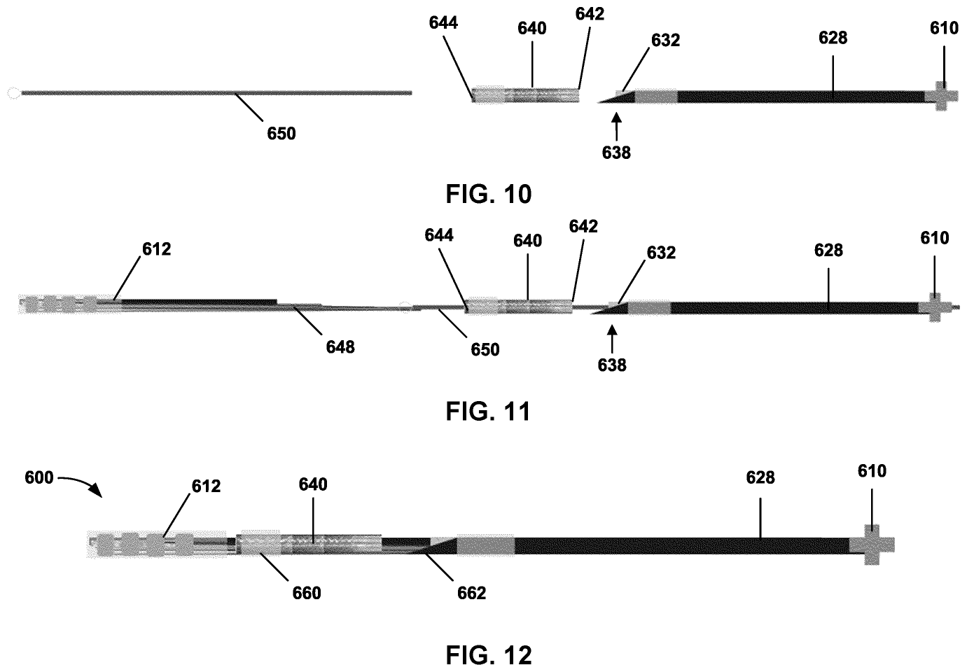

FIG. 10 is a side view of example components of a catheter in an unassembled state, including a proximal hypotube segment 628, and an intermediate segment 640 in accordance with one or more examples of the present disclosure. Proximal hypotube segment 628 may be an example of proximal hypotube segment 128 of FIGS. 1-2. Skived segment 638 may be an example of skived segment 138 of FIGS. 1-2. Electrically insulative tube 632 may be an example of first electrically insulative tube 132 of FIGS. 1-2. Handle 610 may be an example of handle 110 of FIGS. 1-2. Intermediate segment 640 may be an example of intermediate segment 140 of FIGS. 1-2. The components depicted in FIG. 10 are representative of the components described, and depicted in a manner to assist in explanation of assembly of the catheter. The components should not be limited to their visual representations in FIG. 10.

Also shown is attachment mechanism 650. Attachment mechanism 650 may be a lasso, snare, mandrel, or other tool configured to feed or pull electrical leads (e.g., wires 548) of a distal assembly through the lumens of intermediate segment 640 proximal hypotube segment 628, and electrically insulative tube 632. Electrically insulative tube 632 may be located at least partially within an inner lumen of proximal hypotube segment 628. For example, electrically insulative tube 632 may extend distally along a longitudinal axis of proximal hypotube segment 628 into a skived segment 638 of proximal hypotube segment 628. Electrically insulative tube 632 may be attached to proximal hypotube segment 628 at skived segment 628, e.g., using reflow, adhesive, or the like. In other examples, electrically insulative tube 632 may be additionally, or alternatively, attached to hypotube segment 628 at one or more other locations, e.g., at one or more locations of hypotube segment 628 proximal to skived segment 638. In this way, proximal hypotube segment 628 may hold electrically insulative tube 632 rigidly in place during assembly, which may make assembly simpler and less time intensive. Electrically insulative tube 632 may be made of a polymer that is not rigid enough to easily handle by itself, and may kink, rip, roll around, or be crushed during the assembly process. Attaching electrically insulative tube 632 to proximal hypotube segment 628 before feeding electrical leads 648 through electrically insulative tube 632 or proximal hypotube segment 628 prevents kinking, ripping, rolling, and/or crushing of electrically insulative tube 632 during assembly. Intermediate segment 640 may be configured to attach to proximal hypotube segment 628 at a proximal end 642 of intermediate segment 640 via skived segment 638 to form a rapid exchange joint.

In some examples, forming a rapid exchange joint includes a series of steps. For example, a distal portion of skived segment 638 may be inserted into intermediate segment 640, and proximal hypotube segment 628 may be coaxially aligned with intermediate segment 640. A proximal portion of skived segment 638 may not be inserted into intermediate segment 640 such that the uninserted portion of skived segment 638 defines an exchange joint hole in a portion of the shaft of catheter 600. Electrical leads 648 may be fed through proximal hypotube segment 628 and intermediate segment 640 and straightened so that electrical leads 648 lay flat. A proximal portion of a guidewire tube may be inserted through intermediate segment 640 and the exchange joint hole, for example using a mandrel or other tool through an interior of the guidewire tube, such that a proximal end of the guidewire tube may rest on an outer surface of proximal hypotube segment 628, while the portion of the guidewire tube distal to the exchange joint hole is disposed within the lumen of intermediate segment 640. A piece of polymer (e.g., PEBAX®) may be placed over the exchange joint region, and a piece of heat shrink may be placed on top of the polymer. A hot box may be used to reflow the exchange joint region, after which the mandrel and heat shrink may be removed, and the excess polymer material and guidewire tube material may be skive cut to create a smooth outer surface of catheter 600 at the exchange joint.

During assembly, intermediate segment 640 and proximal hypotube segment 628 lined with insulative tube 632 may be aligned with each other (e.g., coaxially) to allow attachment mechanism 650 and electrical leads 648 to easily be fed through the inner lumens of intermediate segment 640, proximal hypotube segment 628, and electrically insulative tube 632. In this way, attachment mechanism 650 may be fed through the coaxially-aligned inner lumens of intermediate segment 640, proximal hypotube segment 628, and electrically insulative tube 632 in one action, rather than first feeding attachment mechanism 650 through electrically insulative tube 632, then feeding attachment mechanism 650 through intermediate segment 640, then feeding attachment mechanism 650 through proximal hypotube segment 628. Attachment mechanism 650 may be positioned within the inner lumens of intermediate segment 640, proximal hypotube segment 628, and electrically insulative tube 632, such that a distal end of attachment mechanism 650 extends distally from distal end 644 of intermediate segment 640 and a proximal end of attachment mechanism 650 extends proximally from a proximal end of proximal hypotube segment 628.

FIG. 11 is a side view of an example partially assembled catheter, including a distal assembly 612 and one or more electrical leads 648, in accordance with one or more examples of the present disclosure. Distal assembly 612 may be an example of neuromodulation element 112 of FIGS. 1-3. Electrical leads 648 may be an example of wires 148 of FIGS. 2-3. Attachment mechanism 650 may be configured feed electrical leads 648 through the inner lumens of intermediate segment 640, proximal hypotube segment 628, and electrically insulative tube 632. Attachment mechanism 650 may attach, at a distal end of attachment mechanism 650, to a proximal end or portion of electrical leads 648, such that attachment mechanism 650 may feed or pull electrical leads 648 through the lumens of intermediate segment 640, proximal hypotube segment 628 and electrically insulative tube 632. In some examples, attachment mechanism 650 is a snare that snares electrical leads 648, before pulling electrical leads 648 through the lumens.

In this way, electrical leads 648 may be fed through the coaxially-aligned inner lumens of intermediate segment 640, proximal hypotube segment 628, and electrically insulative tube 632 in one action, rather than first feeding electrical leads 648 through electrically insulative tube 632, then feeding electrical leads 648 through intermediate segment 640, then feeding electrical leads 648 through proximal hypotube segment 628.

FIG. 12 is a side view of an example assembled catheter 600, in accordance with one or more examples of the present disclosure. Catheter 600 may be an example of catheter 100 of FIGS. 1 and 2. After electrical leads 648 are positioned within the inner lumens of intermediate segment 640 proximal hypotube segment 628, and electrically insulative tube 632, attachment mechanism may be detached from electrical leads 648. Intermediate segment 640 may be attached to proximal hypotube segment 628 via a rapid exchange joint 662. Intermediate segment 640 may be attached to a distal assembly 612 at a distal end 644 of intermediate segment 640 using reflowed polymer, an adhesive, or the like. For example, intermediate segment 640 may be attached to distal assembly 612 or proximal hypotube segment 628 using a polymer 660 (e.g., PEBAX®) reflowed over an outer diameter of intermediate segment 640 and an outer diameter of distal assembly 612 or proximal hypotube segment 628.

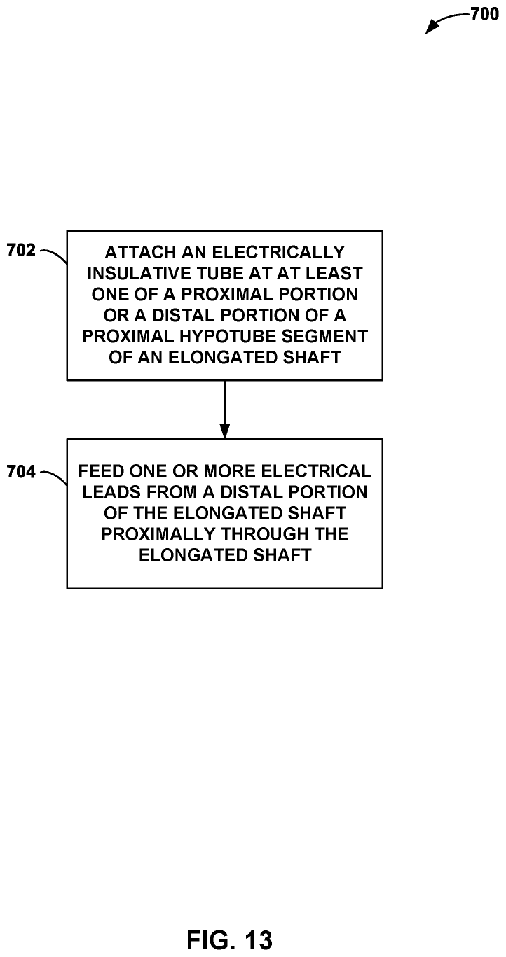

FIG. 13 is a flow chart illustrating an example method 700 for assembling a neuromodulation catheter, in accordance with one or more examples of the present disclosure. For ease of description, the example of FIG. 13 is described with regard to catheter 600 of FIGS. 10-12. However, such an example technique may be employed to assemble any suitable catheter.

Method 700 may include attaching an electrically insulative tube 632 at at least one of a proximal portion or a distal portion of a proximal hypotube segment 628 of an elongate shaft of the catheter 600 (702). Electrically insulative tube 632 may be positioned at least partially within an inner lumen defined by proximal hypotube segment 628. For example, a distal end of electrically insulative tube 632 may be positioned in a distal skived segment 638 of proximal hypotube segment 628 and electrically insulative tube 632 may extend proximally a distance through the inner lumen defined by proximal hypotube segment 628 before terminating at a proximal end within the inner lumen defined by proximal hypotube segment 628. In some examples, a distal end of electrically insulative tube 632 is positioned within the lumen defined by proximal hypotube segment 628 and electrically insulative tube 632 may extend proximally beyond a proximal end of proximal hypotube segment 628. In some examples, a proximal end of electrically insulative tube 632 is positioned within the lumen defined by proximal hypotube segment 628 and electrically insulative tube 632 may extend distally beyond a distal end of proximal hypotube segment 628. In some examples, a distal end of electrically insulative tube 632 is positioned within the inner lumen of proximal hypotube segment 628 proximal of a distal end of proximal hypotube segment 628, and a proximal end of electrically insulative tube 632 is positioned within the inner lumen of proximal hypotube segment 628 distal of a proximal end of proximal hypotube segment 628, such that the entire length of electrically insulative tube 632 is positioned within the inner lumen of proximal hypotube segment 628. In some examples, electrically insulative tube 632 may extend distally along a longitudinal axis of proximal hypotube segment 628 into skived segment 638, and may extend proximally along the longitudinal axis beyond a proximal end of proximal hypotube segment 628.

Electrically insulative tube 632 may be an electrically insulative polymer tube or coating. Electrically insulative tube 632 may define an inner lumen, configured to house one or more electrical leads 648. Electrically insulative tube 632 may electrically insulate electrical leads 648 from proximal hypotube segment 628. When electrically insulative tube 632 is attached to proximal hypotube segment 628, the inner lumen of proximal hypotube segment 628 may extend coaxially with the inner lumen of the insulative tube.

Electrically insulative tube 632 may be attached to proximal hypotube segment 628 using any suitable technique. In some examples, attaching electrically insulative tube 632 to proximal hypotube segment 628 may include attaching electrically insulative tube 632 to a distal portion of proximal hypotube segment 628 with reflowed polymer, adhesive, or the like. For example, with reference to FIG. 4, the electrically insulative tube 232 may extend into a skived segment 238 of the proximal hypotube segment 228, and a proximal outer jacket 230 may be reflowed around a portion of proximal hypotube segment 228 including the skived segment 238 in which the electrically insulative tube 232 extends. In some examples, attaching electrically insulative tube 632 to proximal hypotube segment 628 may include attaching electrically insulative tube 632 to distal skived segment 638 of proximal hypotube segment 628. In some examples, attaching electrically insulative tube 632 to proximal hypotube segment 628 may include attaching electrically insulative tube 632 to a proximal portion of proximal hypotube segment 628 with a reflowed polymer, adhesive, or the like. In some examples, electrically insulative tube 632 may be attached to a proximal portion of handle 610 with a reflowed polymer, adhesive, or the like. In some examples, proximal hypotube segment 628 may define an outer wall, wherein the outer wall includes one or more holes. For example, the outer wall of proximal hypotube segment 628 may be laser-cut with a plurality of holes to add flexibility to proximal hypotube segment 628. Attaching electrically insulative tube 632 to proximal hypotube segment 628 may include using a reflowed polymer over a portion of proximal hypotube segment 628 which includes the one or more holes. The reflowed polymer may sink into the one or more holes and stick to electrically insulative tube 632 positioned within proximal hypotube segment 628.

Attaching electrically insulative tube 632 may include attaching electrically insulative tube 632 to an inner diameter of proximal hypotube segment 628. In some examples, electrically insulative tube 632 may be friction fit onto an inner diameter of the inner lumen of proximal hypotube segment 628. For example, electrically insulative tube 632 may be cooled or otherwise shrunk before being positioned within the inner lumen of proximal hypotube segment 628. After returning to normal temperature while being within the inner lumen of proximal hypotube segment 628, electrically insulative tube 632 may expand to press against inner walls of the inner lumen of proximal hypotube segment 628. In some examples, attaching electrically insulative tube 632 to proximal hypotube segment 628 includes heating electrically insulative tube 632 to melt electrically insulative tube 632 onto an inner diameter of proximal hypotube segment 628. Electrically insulative tube 632 may be melted onto a proximal portion, distal portion, and/or at one or more of a plurality of locations of the inner walls of the inner lumen of proximal hypotube segment 628. In some examples, attaching electrically insulative tube 632 to proximal hypotube segment 628 may include applying an adhesive between an outer diameter of electrically insulative tube 632 and the inner diameter of proximal hypotube segment 628. Method 700 may include applying an adhesive between an outer diameter of electrically insulative tube 632 and the inner diameter of proximal hypotube segment 628 at a proximal portion, distal portion, and/or at one or more of a plurality of locations of the inner walls of the inner lumen of proximal hypotube segment 628. In this way, proximal hypotube segment 628 may hold electrically insulative tube 632 rigidly in place during assembly, and the assembly of neuromodulation catheters may be simpler and less time-intensive.

With reference to FIG. 5, proximal hypotube segment 328 may include a proximal portion a proximal end 304, and a first inner lumen. The catheter may also include proximal assembly 310 defining a second inner lumen, wherein the proximal portion of proximal hypotube segment 328 is received within the second inner lumen of proximal assembly 310, and proximal end 304 of proximal hypotube segment 328 terminates within the second inner lumen.

Attaching electrically insulative tube 632 to proximal hypotube segment 628 may include feeding an attachment mechanism 650 through the inner lumens of intermediate segment 640, proximal hypotube segment 628, and electrically insulative tube 632 before attaching electrically insulative tube 632 to proximal hypotube segment 628. In this way, attachment mechanism 650 may ensure there is an opening within proximal hypotube segment 628 at the portion where electrically insulative tube 632 is attached that electrical leads 648 may be fed through. For example, electrically insulative tube 632 may be attached to proximal hypotube segment 628 by a reflowed polymer over at least a portion of skived segment 638. Attachment mechanism 650 may be positioned within the inner lumens of electrically insulative tube 632 and proximal hypotube segment 628 during the reflow process and may prevent the reflow process from closing off a distal end of the inner lumen of electrically insulative tube 632. The reflowed polymer may flow around attachment mechanism 650, such that when attachment mechanism 650 is removed, an inner lumen of electrically insulative tube 632 remains open at the distal end of electrically insulative tube 632.

Method 700 may also include feeding one or more electrical leads 648 from a distal portion of the elongate shaft proximally through the elongate shaft including through an inner lumen defined by electrically insulative tube 632 and the inner lumen defined by proximal hypotube segment 628 (704). Electrically insulative tube may be made of a polymer that is not rigid enough to easily handle by itself. Attaching electrically insulative tube 632 to proximal hypotube segment 628 before feeding electrical leads through electrically insulative tube 632 or the proximal hypotube may prevent kinking, ripping, rolling, and/or crushing of electrically insulative tube 632 during assembly. In this way also, electrical leads 648 may be fed through the coaxially-aligned inner lumens of intermediate segment 640, proximal hypotube segment 628, and electrically insulative tube 632 in one action, rather than first feeding electrical leads 648 through electrically insulative tube 632, then feeding electrical leads 648 through intermediate segment 640, then feeding electrical leads 648 through proximal hypotube segment 628. Attaching electrically insulative tube 632 to proximal hypotube segment 628 before feeding electrical leads 648 through electrically insulative tube 632 or proximal hypotube segment 628 may also reduce the time of forming an exchange joint between proximal hypotube segment 628 and intermediate segment 640, as electrically insulative tube 632 is bonded or otherwise attached in place so as to prevent shifting of electrically insulative tube 632 during the exchange joint formation.

Feeding one or more electrical leads through the elongate shaft may include attaching a proximal end or portion of electrical leads 648 to a distal end of attachment mechanism 650, such that attachment mechanism 650 may feed or pull electrical leads 648 proximally through the lumens of intermediate segment 640, proximal hypotube segment 628, and electrically insulative tube 632. Feeding one or more electrical leads 648 through the elongate shaft may include pulling electrical leads 648 through portions of the elongate shaft via attachment mechanism 650. Attachment mechanism 650 may be a lasso, snare, mandrel, or other tool configured to feed or pull electrical leads 648 of a distal assembly 612 through the inner lumens of intermediate segment 640, proximal hypotube segment 628, and electrically insulative tube 632. In some examples, the proximal end of electrical leads 648 may be fed proximally through the inner lumens of the elongate shaft. In some examples, a distal end of electrical leads 648 may be fed distally through the inner lumens of the elongate shaft. For example, a proximal end of attachment mechanism 650 may be attached to a distal end or portion of electrical leads 648, and attachment mechanism may feed or pull electrical leads 648 distally through the lumens of intermediate segment 640, proximal hypotube segment 628, and electrically insulative tube 632.

After feeding electrical leads 648 through the inner lumens of the elongate shaft, method 700 may include detaching attachment mechanism 650 from electrical leads 648. Method 700 may also include attaching distal end 644 of intermediate segment 640 to distal assembly 612 of the distal portion of the elongate shaft, and attaching proximal end 642 of intermediate segment 640 to a distal portion of proximal hypotube segment 628. Intermediate segment 640 may be attached to distal assembly 612 using reflowed polymer, an adhesive, or the like. For example, intermediate segment 640 may be attached to distal assembly 612 using Pebax®. Attaching intermediate segment 640 to proximal hypotube segment 628 may include forming a rapid exchange joint at skived segment 638 between intermediate segment 640 of the elongate shaft and proximal hypotube segment 628. In addition to, or instead of, PEBAX®, intermediate segment 640 may be attached to distal assembly 612 or proximal hypotube segment 628 using another reflowable polymer.

In some examples, a proximal end of a guidewire tube may be positioned in the rapid exchange joint to allow a guidewire to pass from the inside of the elongate shaft distal to the rapid exchange joint to the outside of the elongate shaft proximal of the rapid exchange joint. A proximal portion of the guidewire tube, including the proximal end of the guidewire tube may be attached to the elongate shaft using reflowed polymer, an adhesive, or the like. The proximal portion of the guidewire tube may include a skived section configured to define a smooth diameter transition between the proximal outer jacket (e.g., a polymer outer jacket) and a location where a portion of the guidewire tube extends into the rapid exchange joint. The guidewire may be positioned inside an interior lumen of the guidewire tube in a distal portion of the elongate shaft, exit the elongate shaft at the rapid exchange joint, and be positioned outside the elongate shaft from the rapid exchange joint proximally of the rapid exchange joint. The guidewire tube may extend distally from the rapid exchange joint to the distal assembly.

In some examples the catheter may not include a rapid exchange joint. In these examples, the guidewire tube may be disposed within the inner lumen of catheter 600 from distal assembly 612 to at least handle 610. In some examples, the guidewire tube may be disposed inside electrically insulative tube 632. In some examples, the guidewire tube may travel parallel to electrically insulative tube 632 inside one or more segments of catheter 600. In some examples, electrically insulative tube 632 may be attached to proximal hypotube segment 628 on only a portion of an outer diameter of electrically insulative tube 632.

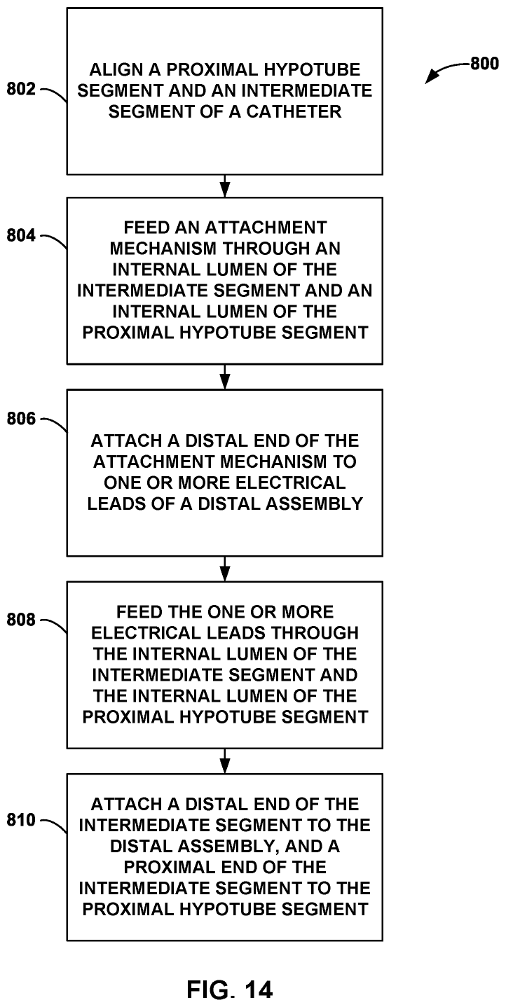

FIG. 14 is a flow chart illustrating another example method 800 for assembling a neuromodulation catheter, in accordance with one or more examples of the present disclosure. For ease of description, the example method 800 of FIG. 14 is described with regard to catheter 600 of FIGS. 10-12.

Method 800 may include coaxially aligning proximal hypotube segment 628 and intermediate segment 640 of catheter 600, where proximal hypotube segment 628 contains electrically insulative tube 632 within an inner lumen of proximal hypotube segment 628 (802). Coaxially aligning intermediate segment 640 and proximal hypotube segment 628 may allow attachment mechanism 650 and electrical leads 648 to easily be fed through the inner lumens of intermediate segment 640, proximal hypotube segment 628, and electrically insulative tube 632.

Method 800 may also include feeding attachment mechanism 650 through the internal lumens of intermediate segment 640, proximal hypotube segment 628, and electrically insulative tube 632 (804). Attachment mechanism 650 may be positioned within the inner lumens of intermediate segment 640, proximal hypotube segment 628, and electrically insulative tube 632 such that a distal end of attachment mechanism 650 extends distally from distal end 644 of intermediate segment 640 and such that a proximal end of attachment mechanism 650 extends proximally from a proximal end of proximal hypotube segment 628.

Method 800 may further include attaching a distal end of attachment mechanism 650 to one or more electrical leads 648 of a distal assembly of the catheter (806). Attachment mechanism 650 may attach at a distal end of attachment mechanism 650 to a proximal end or proximal portion of electrical leads 648, such that attachment mechanism 650 may feed or pull electrical leads 648 through the lumens of intermediate segment 640, proximal hypotube segment 628, and electrically insulative tube 632. For example, attachment mechanism 650 may be a lasso, snare, mandrel, or other tool configured to feed or pull electrical leads 648 of distal assembly 612 through the lumens of intermediate segment 640, proximal hypotube segment 628, and electrically insulative tube 632.

Method 800 may also include feeding the one or more electrical leads 648 through the internal lumen of intermediate segment 640, proximal hypotube segment 628, and electrically insulative tube 632 (808). Feeding the one or more electrical leads 648 through the internal lumens may include pulling attachment mechanism 650 such that the distal end of attachment mechanism 650 that is attached to the proximal end or proximal portion of electrical leads 648 is guided all the way through the internal lumens described above. The proximal ends of one or more of electrical leads 648 may extend further proximally than a proximal end of proximal hypotube segment 628, or a proximal end of electrically insulative tube 632. After guiding electrical leads 648 all the way through the elongate shaft, method 800 may include detaching attachment mechanism 650 from electrical leads 648.

Method 800 may further include attaching distal end 644 of intermediate segment 640 to distal assembly 612, and proximal end 642 of intermediate segment 640 to proximal hypotube segment 628 (810). Intermediate segment 640 may be attached to distal assembly 612 using reflowed polymer, an adhesive, or the like. For example, intermediate segment 640 may be attached to distal assembly 612 using Pebax® bond-aids. Attaching intermediate segment 640 to proximal hypotube segment 628 may include forming a rapid exchange joint at skived segment 638 between intermediate segment 640 of the elongate shaft and proximal hypotube segment 628.

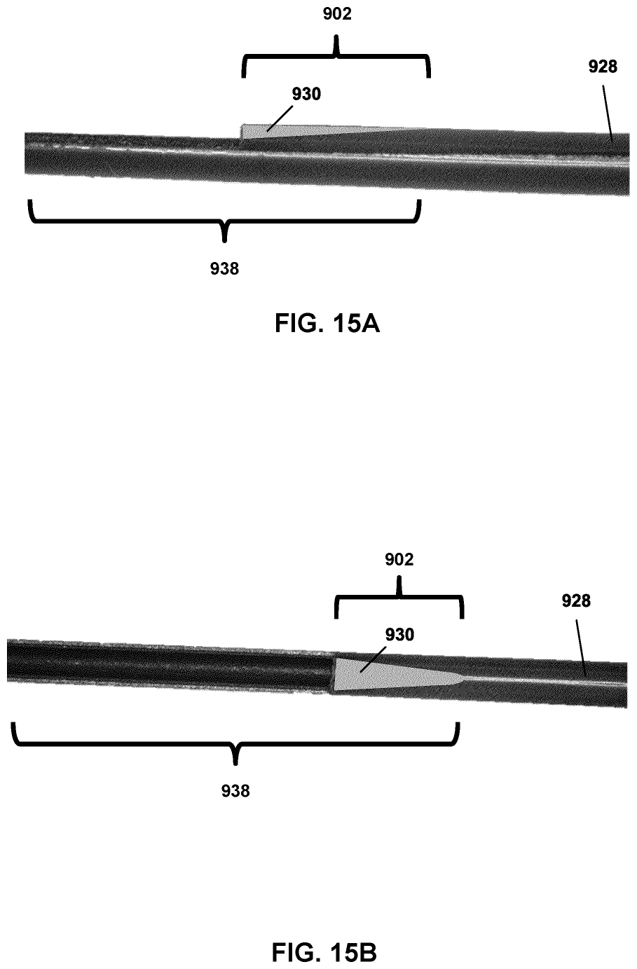

FIGS. 15A and 15B are example side views of a distal portion of proximal hypotube segment 928 and proximal outer jacket 930, in accordance with one or more examples of the present disclosure. Proximal hypotube segment 928 may be an example of proximal hypotube segment 128 of FIGS. 1-2. Skived segment 938 may be an example of skived segment 138 of FIGS. 1-2. Proximal outer jacket 930 may be an example of proximal outer jacket 130 of FIGS. 1-2. Proximal outer jacket 930 may include a distal portion 902 that extends a distance into distal skived segment 938 of proximal hypotube segment 928. A distal portion of an electrically insulative tube may extend a similar distance into distal skived segment 928, such that proximal outer jacket 930 and the electrically insulative tube may be attached to one another and proximal hypotube segment 928 via reflow, adhesive, or the like. A length of the distal portion 902 may be configured to allow room for optimal threading and exchange joint bond alignment. A distal portion 902 that is too long may not allow enough room for electrical wires to be fed through the inner lumen of the proximal hypotube segment and electrically insulative tube, and may become crushed upon formation of the rapid exchange joint.

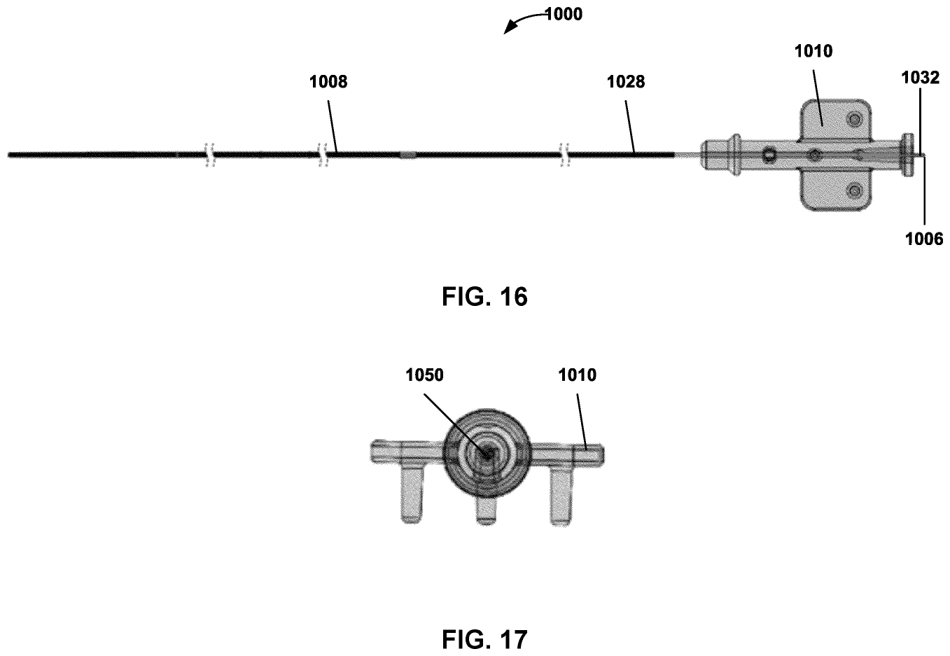

FIG. 16 is an example side view of a portion of neuromodulation catheter 1000, including an elongate shaft 1008 and proximal assembly 1010, in accordance with one or more examples of the present disclosure. Catheter 1000 may be an example of catheter 100 of FIGS. 1 and 2. Elongate shaft 1008 may be an example of elongate shaft 108 of FIGS. 1 and 2. Proximal hypotube segment 1028 may be an example of proximal hypotube segment 128 of FIGS. 1-2. Electrically insulative tube 1032 may be an example of first electrically insulative tube 132 of FIGS. 1-2. Proximal assembly 1010 may be an example of handle 110 of FIGS. 1 and 2. Proximal assembly 1010 may define an inner lumen, wherein a proximal portion of proximal hypotube segment 1028 may be received within the inner lumen of proximal assembly 1010, and a first proximal end of proximal hypotube segment 1028 terminates within the inner lumen. A second proximal end 1006 of electrically insulative tube 1032 may extend proximally of proximal assembly 1010 beyond a third proximal end of the inner lumen defined by proximal assembly 1010. The portion of electrically insulative tube 1032 proximal to proximal assembly 1010 may act to protect electrical leads and/or thermocouple wires located within proximal assembly 1010.

FIG. 17 is a side view of example proximal assembly 1010 of FIG. 16, in accordance with one or more examples of the present disclosure. Proximal assembly 1010 may define an inner lumen 1050. A proximal end of proximal hypotube segment 1028 includes a proximal portion and a proximal end, wherein the proximal portion of proximal hypotube segment 1028 is received within inner lumen 1050 of proximal assembly 1010, and wherein the proximal end of proximal hypotube segment 1028 terminates within inner lumen 1050.

Catheters configured in accordance with at least some examples of the present technology may be well suited (e.g., with respect to sizing, flexibility, operational characteristics, and/or other attributes) for performing renal neuromodulation in human patients. Renal neuromodulation is the partial or complete incapacitation or other effective disruption of nerves of the kidneys (e.g., nerves terminating in the kidneys or in structures closely associated with the kidneys). In particular, renal neuromodulation may include inhibiting, reducing, and/or blocking neural communication along neural fibers (e.g., efferent and/or afferent neural fibers) of the kidneys. Such incapacitation may be long-term (e.g., permanent or for periods of months, years, or decades) or short-term (e.g., for periods of minutes, hours, days, or weeks). Renal neuromodulation is expected to contribute to the systemic reduction of sympathetic tone or drive and/or to benefit at least some specific organs and/or other bodily structures innervated by sympathetic nerves. Accordingly, renal neuromodulation is expected to be useful in treating clinical conditions associated with systemic sympathetic overactivity or hyperactivity, particularly conditions associated with central sympathetic overstimulation. For example, renal neuromodulation is expected to efficaciously treat hypertension, heart failure, acute myocardial infarction, metabolic syndrome, insulin resistance, diabetes, left ventricular hypertrophy, chronic and end stage renal disease, inappropriate fluid retention in heart failure, cardio-renal syndrome, polycystic kidney disease, polycystic ovary syndrome, osteoporosis, erectile dysfunction, and sudden death, among other conditions.

Renal neuromodulation may be electrically-induced, thermally-induced, or induced in another suitable manner or combination of manners at one or more suitable treatment locations during a treatment procedure. The treatment location may be within or otherwise proximate to a renal lumen (e.g., a renal artery, a ureter, a renal pelvis, a major renal calyx, a minor renal calyx, or another suitable structure), and the treated tissue may include tissue at least proximate to a wall of the renal lumen. For example, with regard to a renal artery, a treatment procedure may include modulating nerves in the renal plexus, which lay intimately within or adjacent to the adventitia of the renal artery. Various suitable modifications may be made to the catheters described above to accommodate different treatment modalities. For example, the band electrodes 204 may be replaced with transducers to facilitate transducer-based treatment modalities.

Renal neuromodulation may include an electrode-based treatment modality alone or in combination with another treatment modality. Electrode-based treatment may include delivering electricity and/or another form of energy to tissue at or near a treatment location to stimulate and/or heat the tissue in a manner that modulates neural function. For example, sufficiently stimulating and/or heating at least a portion of a sympathetic renal nerve may slow or potentially block conduction of neural signals to produce a prolonged or permanent reduction in renal sympathetic activity. A variety of suitable types of energy may be used to stimulate and/or heat tissue at or near a treatment location. For example, neuromodulation in accordance with examples of the present technology may include delivering RF energy and/or another suitable type of energy. An electrode used to deliver this energy may be used alone or with other electrodes in a multi-electrode array.

Heating effects of electrode-based treatment may include ablation and/or non-ablative alteration or damage (e.g., via sustained heating and/or resistive heating). For example, a treatment procedure may include raising the temperature of target neural fibers to a target temperature above a first threshold to achieve non-ablative alteration, or above a second, higher threshold to achieve ablation. The target temperature may be higher than about body temperature (e.g., about 37° C.) but less than about 45° C. for non-ablative alteration, and the target temperature may be higher than about 45° C. for ablation. Heating tissue to a temperature between about body temperature and about 45° C. may induce non-ablative alteration, for example, via moderate heating of target neural fibers or of luminal structures that perfuse the target neural fibers. In cases where luminal structures are affected, the target neural fibers may be denied perfusion resulting in necrosis of the neural tissue. Heating tissue to a target temperature higher than about 45° C. (e.g., higher than about 60° C.) may induce ablation, for example, via substantial heating of target neural fibers or of luminal structures that perfuse the target fibers. In some patients, it may be desirable to heat tissue to temperatures that are sufficient to ablate the target neural fibers or the luminal structures, but that are less than about 90° C. (e.g., less than about 85° C., less than about 80° C., or less than about 75° C.).

This disclosure is not intended to be exhaustive or to limit the present technology to the precise forms disclosed herein. Although specific examples are disclosed herein for illustrative purposes, various equivalent modifications are possible without deviating from the present technology, as those of ordinary skill in the relevant art will recognize. In some cases, well-known structures and functions have not been shown and/or described in detail to avoid unnecessarily obscuring the description of the examples of the present technology. Although steps of methods may be presented herein in a particular order, in alternative examples the steps may have another suitable order. Similarly, certain aspects of the present technology disclosed in the context of particular examples may be combined or eliminated in other examples. Furthermore, while advantages associated with certain examples may have been disclosed in the context of those examples, other examples may also exhibit such advantages, and not all examples need necessarily exhibit such advantages or other advantages disclosed herein to fall within the scope of the present technology. Accordingly, this disclosure and associated technology may encompass other examples not expressly shown and/or described herein.

The methods disclosed herein include and encompass, in addition to methods of practicing the present technology (e.g., methods of making and using the disclosed devices and systems), methods of instructing others to practice the present technology.

Throughout this disclosure, the singular terms “a,” “an,” and “the” include plural referents unless the context clearly indicates otherwise. Similarly, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the terms “comprising” and the like are used throughout this disclosure to mean including at least the recited feature(s) such that any greater number of the same feature(s) and/or one or more additional types of features are not precluded. Directional terms, such as “upper,” “lower,” “front,” “back,” “vertical,” and “horizontal,” may be used herein to express and clarify the relationship between various elements. It should be understood that such terms do not denote absolute orientation. Reference herein to “one example,” “an example,” or similar formulations means that a particular feature, structure, operation, or characteristic described in connection with the example may be included in at least one example of the present technology. Thus, the appearances of such phrases or formulations herein are not necessarily all referring to the same example. Furthermore, various particular features, structures, operations, or characteristics may be combined in any suitable manner in one or more examples of the present technology.

Various aspects of the disclosure have been described. These and other aspects are within the scope of the following claims.

1. A catheter system comprising:
an elongate shaft including a distal portion and a proximal portion;
a neuromodulation element on the distal portion of the elongate shaft, wherein the proximal portion of the elongate shaft includes:
a proximal hypotube segment defining an inner lumen, and
an electrically insulative tube located at least partially within the inner lumen of the proximal hypotube segment, the electrically insulative tube being attached to the proximal hypotube segment at at least one of a proximal portion or a distal portion of the proximal hypotube segment; and
one or more electrical leads extending distally within the inner lumen of the proximal hypotube segment and the electrically insulative tube to the neuromodulation element on the distal portion of the elongate shaft.

2. The catheter system of claim 1, wherein the proximal hypotube segment comprises a distal skived segment,
wherein the electrically insulative tube extends distally along a longitudinal axis of the proximal hypotube segment into the skived segment, and
wherein the electrically insulative tube extends proximally along the longitudinal axis beyond a proximal end of the proximal hypotube segment.

3. The catheter system of claim 2, wherein the electrically insulative tube is attached to the distal skived segment with a reflowed polymer.

4. The catheter system of claim 1,
wherein the proximal hypotube segment comprises the proximal portion and a proximal end, and the inner lumen comprises a first inner lumen,
the catheter further comprising a proximal assembly defining a second inner lumen,
wherein the proximal portion of the proximal hypotube segment is received within the second inner lumen of the proximal assembly, and
wherein the proximal end of the proximal hypotube segment terminates within the second inner lumen.

5. The catheter system of claim 1, wherein the electrically insulative tube is attached at the distal portion of the proximal hypotube segment with a reflowed polymer.

6. The catheter system of claim 1, wherein the electrically insulative tube is friction fit onto an inner diameter of the proximal hypotube segment.

7. The catheter system of claim 1, wherein the electrically insulative tube is melted onto an inner diameter of the proximal hypotube segment to attach the electrically insulative tube to the proximal hypotube segment.

8. The catheter system of claim 1, wherein an adhesive is applied between an outer diameter of the electrically insulative tube and an inner diameter of the proximal hypotube segment to attach the electrically insulative tube to the proximal hypotube segment.

9. The catheter system of claim 1, wherein
the distal portion of the elongate shaft comprises a distal assembly,
the elongate shaft further comprises an intermediate segment, wherein the intermediate segment is attached at one end to the distal assembly and attached at the other end to the proximal hypotube segment via a rapid exchange joint, and
the one or more electrical leads extend distally within an inner lumen of the intermediate segment to the neuromodulation element on the distal portion of the elongate shaft.

10. The catheter system of claim 9, wherein the proximal hypotube segment and the intermediate segment comprise steel or Nitinol.

11. A method of assembling a catheter, the method comprising:
attaching an electrically insulative tube at at least one of a proximal portion or a distal portion of a proximal hypotube segment of an elongate shaft of the catheter, wherein the electrically insulative tube is located at least partially within an inner lumen defined by the proximal hypotube segment; and
feeding one or more electrical leads from a distal portion of the elongate shaft proximally through the elongate shaft, including through an inner lumen defined by the electrically insulative tube and the inner lumen defined by the proximal hypotube segment.

12. The method of claim 11, wherein the proximal hypotube segment comprises a distal skived segment, and attaching the electrically insulative tube to the proximal hypotube segment comprises attaching the electrically insulative tube to the skived segment with reflowed polymer.

13. The method of claim 12, further comprising forming an exchange joint at the skived segment between an intermediate segment of the elongate shaft and the proximal hypotube segment.

14. The method of claim 12,
wherein the electrically insulative tube extends distally along a longitudinal axis of the proximal hypotube segment into the skived segment, and
wherein the electrically insulative tube extends proximally along the longitudinal axis beyond a proximal end of the proximal hypotube segment.

15. The method of claim 11, wherein the proximal hypotube segment comprises the proximal portion and a proximal end, and the inner lumen comprises a first inner lumen, the method further comprising:
introducing the proximal portion of the proximal hypotube segment within a second inner lumen of a proximal assembly such that the proximal end of the proximal hypotube segment terminates within the second inner lumen.

16. The method of claim 11, wherein attaching the electrically insulative tube comprises attaching the electrically insulative tube to the distal portion of the proximal hypotube segment with a reflowed polymer.

17. The method of claim 11, wherein attaching the electrically insulative tube comprises attaching the electrically insulative tube to an inner diameter of the proximal hypotube segment.

18. The method of claim 11, wherein attaching the electrically insulative tube comprises heating the electrically insulative tube to melt the electrically insulative tube onto an inner diameter of the proximal hypotube segment.

19. The method of claim 11, wherein attaching the electrically insulative tube comprises applying an adhesive between an outer diameter of the electrically insulative tube and an inner diameter of the proximal hypotube segment.

20. The method of claim 11, further comprising attaching a distal end of an intermediate segment of the elongate shaft to a distal assembly of the distal portion of the elongate shaft.
